(19) United States
(12) Patent Application Publication
(10) Pub. No.: US 2018/0147092 A1
(43) Pub. Date: May 31, 2018

(54) SUBDERMAL POCKET COMPRESSION APPARATUS

(71) Applicant: Seville Medical Inc., Toronto (CA)

(72) Inventor: Robert Krensky, Toronto (CA)

(21) Appl. No.: 15/823,908

(22) Filed: Nov. 28, 2017

Related U.S. Application Data
(60) Provisional application No. 62/426,828, filed on Nov. 28, 2016.

(51) Int. Cl. A61F 13/02; A61F 5/34

(52) U.S. Cl. CPC A61F 13/0216; A61B 17/135; A61F 5/34

(57) ABSTRACT

A compression apparatus for a wound, the apparatus having: a base comprising a base opening with access to the wound; a housing removably attached to the base; an inflatable member within the housing; wherein the inflatable member is fillable with an inflation medium such that the inflatable member expands within the housing opening and expands through the base opening to apply a compressive force on the wound.

### CROSS-REFERENCE TO RELATED APPLICATIONS

This application claims the benefit of priority to U.S. Provisional Application Ser. No. 62/426,828, filed on Nov. 28, 2016.

### FIELD OF THE INVENTION

The present invention relates to a compression apparatus for wounds.

### DESCRIPTION OF THE RELATED ART

Following certain surgical procedures, it is often desirable or recommended to apply pressure to a localized surgical site on a patient during the post-operative period to accelerate healing and minimize complications. For example, permanent pacemakers and implantable cardioverter defibrillators (ICD) are implantable medical devices that treat a variety of heart conditions and arrhythmias. Generally, procedures pertaining to these implantable medical devices are typically performed on an out-patient basis, and one potential complication includes hematoma formation within the subdermal pocket formed below the skin, which, if left untreated, could lead to an infection which may require the patient to return to the operating room or have a lengthier hospital stay. It has been reported that the risk of developing a hematoma post-pacemaker insertion is between 2% to 10%, and the probability increases for those patients taking antiplatelet or anticoagulant medication which is routinely prescribed in certain cardiac patients.

The best practice to minimize hematoma formation requires taking preventative precautions, such as applying direct compression over the subdermal pocket during the post-operative period. Various methods for applying direct compression over the subdermal pocket have been proposed. Some of these methods include sand bags, straps, pads and various contraptions that apply pressure from front and back of the patient either across the underarm area or over the shoulder. However, these methods employ devices that are often cumbersome, can easily slip out of position, frequently impede mobility, and some even require the patient to remain supine for long periods of time.

It is an object of the present invention to mitigate or obviate at least one of the above-mentioned disadvantages.

### SUMMARY OF THE INVENTION

In one of its aspects, there is provided a compression apparatus for a wound, the apparatus comprising:

a housing;

an inflatable member within the housing;

a base comprising a base opening with access to the wound; the base being removably attached to the housing;

wherein the inflatable member is fillable with a fluid such that the inflatable member expands within the housing opening and expands through the base opening to apply a compressive force on the wound.

In another of its aspects, there is provided a compression apparatus for a wound, the apparatus comprising:

a base comprising a base opening with access to the wound; wherein the base comprises a peripheral portion defining the base opening and a bottom surface with an adhesive backing to removably attach the base around the wound such that the wound is located within the base opening;
a housing comprising a top wall with a side wall depending therefrom to define a housing opening, and a flange extending from the side wall; and
an inflatable member within the housing; wherein the inflatable member is fillable with an inflation medium such that the inflatable member expands within the housing opening and expands through the base opening to apply a compressive force on the wound.

In yet another of its aspects, there is provided a method of applying a compression force on a wound, the method comprising:
providing a housing with an inflatable member within the housing, wherein the housing comprises a base with base opening for access to the wound;
removably attaching the base around the wound, wherein the base comprises an adhesive backing; and
filling the inflatable member with an inflation medium such that the inflatable member expands within housing and expands through the base opening to apply the compressive force on the wound.

Advantageously, compared to the prior art methods, the compression apparatus is a low cost, compact device that fits over the subdermal pocket area and allows for an internal chamber containing an inflatable member that can be inflated and deflated to provide direct compression to a post-operative surgical site. In addition, the compression apparatus does not require straps or harnesses for securement on the patient.

### BRIEF DESCRIPTION OF THE DRAWINGS

Several exemplary embodiments of the present invention will now be described, by way of example only, with reference to the appended drawings in which:

FIG. 1 is a cut away perspective view of a compression apparatus, in one embodiment;

FIG. 2 shows a sectional view of the compression apparatus of FIG. 1;

FIG. 3 shows the compression apparatus over a wound on an anatomical structure;

FIG. 4a shows a perspective view of a compression apparatus, in another embodiment;

FIG. 4b shows a sectional view of the compression apparatus of FIG. 4a;

FIGS. 5a and 5b show perspective views of a compression apparatus, in yet another embodiment; and

FIG. 6 shows the compression apparatus in use on a patient.

### DETAILED DESCRIPTION OF EXEMPLARY EMBODIMENTS

The detailed description of exemplary embodiments of the invention herein makes reference to the accompanying block diagrams and schematic diagrams, which show the exemplary embodiment by way of illustration. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented.

Moreover, it should be appreciated that the particular implementations shown and described herein are illustrative of the invention and its best mode and are not intended to otherwise limit the scope of the present invention in any way. Connecting lines shown in the various figures contained herein are intended to represent exemplary functional relationships and/or physical couplings between the various elements. It should be noted that many alternative or additional functional relationships or physical connections may be present in a practical system.

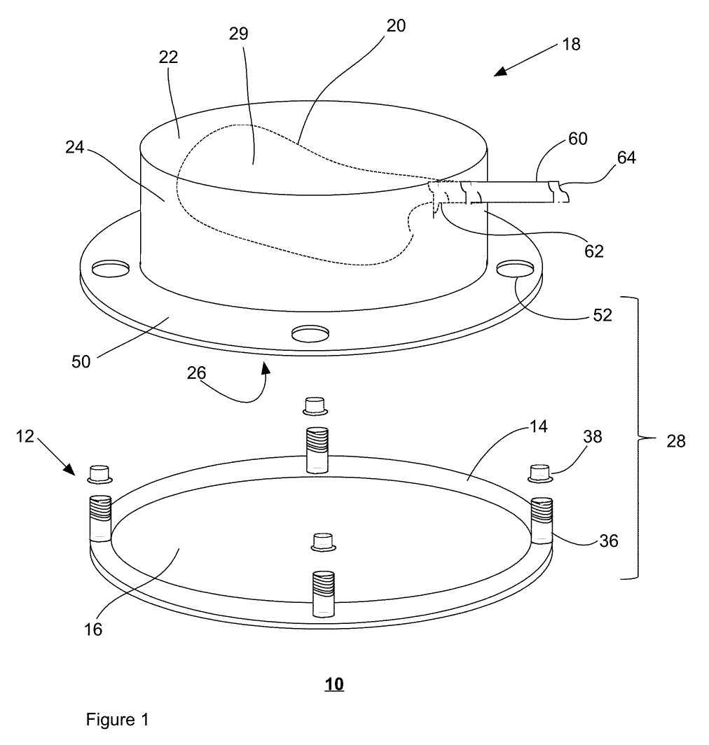

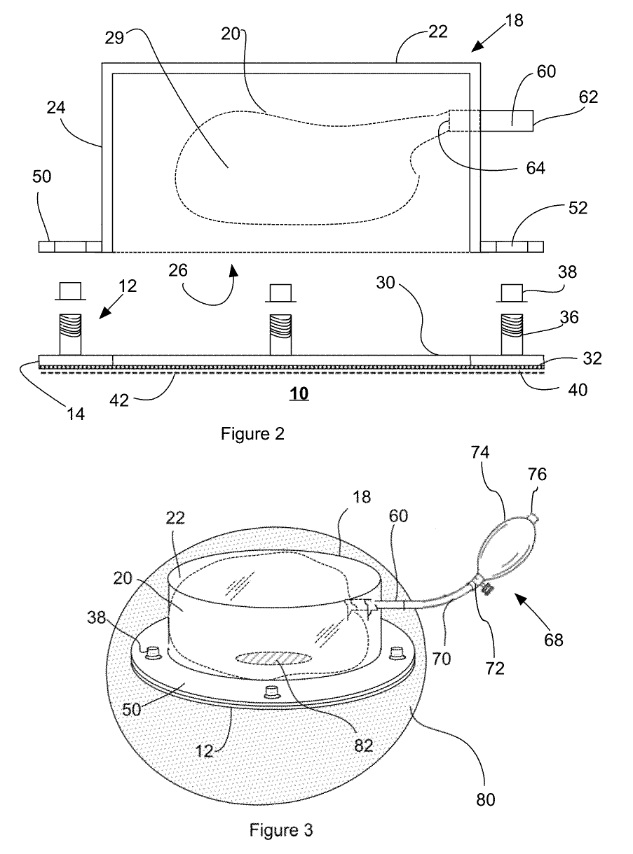

FIGS. 1 and 2 show an exemplary compression apparatus for a wound, generally identified by reference numeral 10, having base 12 comprising peripheral portion 14 defining base opening 16, and housing 18 containing inflatable member 20. Generally, inflatable member 20 may be any balloon-type structure capable of expanding and contracting, and may be an elastomeric or polymeric structure such as an inflatable bladder. Preferably, inflatable bladder 20 is non-porous, and washable. Housing 18 comprises top wall 22 and sidewall 24 defining housing opening 26 opposite top wall 22, and housing 18 is removably attached to base 12 by a fastening means 28. In one exemplary implementation, base 12 is secured on a patient such that peripheral portion 14 surrounds the wound, such that the wound is located within base opening 16; and housing 18 with inflatable bladder 20 is secured to base 12. Plenum 29 of inflatable bladder 20 is filled with an inflation medium such that inflatable bladder 20 expands through housing opening 26 and base opening 16 to apply a compressive force on the wound. Inflatable bladder 20 is inflated to a pressure suitable to minimize hematoma formation by providing a sufficient compressive force on the wound, while also minimizing any local clinical or mechanical issues.

Peripheral portion 14 of base 12 comprises top surface 30 opposite to bottom surface 32. Extending from top surface 30 is one portion of fastening means 28, such as threaded posts 36, which receive screw elements 38. Bottom surface 32 includes adhesive layer 40 with release liner 42 for removal before use. Preferably, adhesive layer 40 is skin-friendly and has bond strength sufficient to withstand the compressive forces imparted by inflatable bladder 20, and housing 18 via fastening means 28 which may provide supplementary pressure on inflatable bladder 20, as will be explained later.

Housing 18 further comprises flange 50 extending from sidewall 24 adjacent to housing opening 26. Flange 50 includes apertures 52 for receiving threaded posts 36, such that screw elements 38 are fastened thereon to sandwich flange 50 between screw elements 38 and top surface 30 of base 12. Sidewall 24 also includes port 60 extending therethrough with one end 62 coupled to inflatable bladder 20 and another end 64 coupled to a source of inflation medium 66. End 64 may comprise a connector for coupling port 60 to a fluid source, or pump. The connector may include a valve mechanism for permitting inflation or deflation of inflatable bladder 20 by setting the valve mechanism at one of two positions. The first valve mechanism position connects end 64 to the source of inflation medium 66 such that the bladder inflates to place inflatable bladder 20 in an inflated condition, while a second valve mechanism position allows the inflation medium to escape via end 64 such that the bladder deflates and collapses, thus placing inflatable bladder 20 in a deflated condition.

Looking at FIG. 3, after the application area, or post-surgical site 80, on the patient has been prepared e.g. removal of hair, compression apparatus 10 can be removably attached onto the patient. Following the removal of the release liner 42 to expose adhesive layer 40, base 12 is secured to an area of interest 80 on a patient, such as a surgical wound. Inflatable bladder 20 is coupled to end 62 of port 60, and housing 18 is placed over base 12 such that threaded posts 36 are received by apertures 52 in flange 50 of housing 18. Flange 50 rests on top surface 30 of base 12, and screw elements 38 are rotatably attached to threaded posts 36 and advance towards flange 50 to hold base 12 and housing 18 together. A source of inflation medium 66, such as pump 68 is coupled to end 64 of port 60. With air as the chosen inflation medium 66, pump 68 is preferably formed from an elastomeric or latex-free rubber material and includes a connecting conduit 70 connected via a release valve 72 to a squeeze bulb 74. In operation, air enters opening 76 of squeeze bulb 74 and is prevented from leaving through that opening because of an internal one-way valve at opening 76. Squeezing of bulb 74 forces the air therein to exit bulb 74 and travel along conduit 70, via port 60 into plenum 29 of inflatable bladder 20. Another internal one-way valve precludes that air from returning to bulb 74 when bulb 74 is released.

In order to conform to the nature of the part of the anatomy with area of interest 80 for treatment, such as wound 82, base 12 and flange 50 are made from a pliable material. Accordingly, the flexibility of base 12 enables releasable engagement with the complex contours of the anatomical structure. As inflatable bladder 20 expands it presses up against top wall 22 of housing 18 and presses down against wound 82 to apply a compressive force thereon. Typically, a protective layer, such as a wound dressing, gauze or bandage, may be placed on wound 82. Alternatively, at the discretion of the healthcare professional, wound 82 may be left exposed.

To deflate bladder 20, release valve 72 is opened and the air returns along conduit 70 to exit to the atmosphere from release valve 72. In order to facilitate dressing changes or bathing, housing 18 may be removed from base 12 by unscrewing screw elements 38 from threaded posts 36 and removing housing 18 from base 12, such that flange 50 is clear of threaded posts 36. The skin-adhesive side 32 of the base 12 remains in place on site 80 of anatomical structure, however, should it fall off due to weakened bond strength then an adhesive coating may be applied to bottom surface 32, as needed, or a double-sided self-adhesive tape backing 40 may be applied. The outside surface of inflatable bladder 20 may be cleaned as necessary.

In another exemplary implementation, bottom surface 32 includes adhesive sheet with one side adhered to bottom surface 32, and opposing side having adhesive layer 40 with release liner 42 for removal before use.

In another exemplary implementation, adhesive layer 40 is able to maintain its bonding properties for prolonged time periods while on the patient's skin.

In another exemplary implementation, adhesive layer 40 is skin-friendly, breathable, allows residue free removal, and imparts minimal pain to the patient upon removal.

In another exemplary implementation, inflatable bladder 20 is filled with inflation medium 66 to a predetermined pressure. Accordingly, release valve 72 may include a pressure gauge. As an example, the pressure gauge may include indicia to show the inflated pressure of inflatable bladder 20, and or a desired operating pressure. For example, the gauge may include a desired operating pressure marker. In one example, a pressure of approximately 0.2 psi is desirable to minimize hematoma formation, and therefore the pressure marker may be indicia corresponding to 0.2 psi. The gauge may also include indicia corresponding to over-inflation which may result in undesirable, excessive compressive forces on wound 82 which may impede the healing process, or indicia corresponding to under-inflation which may result in undesirable, non-effective compressive forces on wound 82.

In another exemplary implementation, fastening means 28 comprises at least one of a quick release mechanism for housing 18, a clamping mechanism, and Velcro™.

In another exemplary implementation, fastening means 28 comprises threaded apertures in base 12 for receiving suitably threaded screws to releasably secure housing 18 onto base 12.

In another exemplary implementation, fastening means 28 is adjustable to regulate the compression of inflatable bladder 20 by positioning top wall 22 and flange 50 of housing 18 closer to base 12, or away from base 12. For instance, when top wall 22 and flange 50 of housing 18 are caused to advance towards base 12, then additional compressive forces are imparted onto inflatable bladder 20, thereby increasing the overall compression on the surgical site. On the other hand, when top wall 22 and flange 50 of housing 18 are caused to retreat from base 12, then compressive forces are removed from inflatable bladder 20, thereby reducing the overall compression on the surgical site.

In another exemplary implementation, housing 18 comprises a lower housing portion and an upper housing portion removably attached to lower housing portion. The lower housing portion comprises a sidewall defining a top opening and a bottom opening. The lower housing portion includes a flange similar to flange 50 with apertures. The upper housing portion comprises a sidewall depending from a top wall, with an opening opposite the top wall. In one example, the upper housing portion is rotatably affixed to the lower housing portion such that the top wall abuts inflatable bladder 20 and causes compression of inflatable bladder 20. Accordingly, the compressive forces on wound 82 may be controlled by the position of the upper portion. For instance, when the top wall is caused to advance towards the top opening of lower housing portion, then additional compressive forces are imparted onto inflatable bladder 20, thereby increasing the overall compression on the surgical site. On the other hand, when the top wall is caused to retreat from the top opening of lower housing portion, then compressive forces are removed from inflatable bladder 20, thereby reducing the overall compression on the surgical site. In one example, the upper housing portion is rotated onto the lower housing portion 90. In another example, the upper housing portion is rotated onto the lower housing portion by pressing a button on the housing 18 that allows rotation of the upper housing portion while pressed. Rotation stops once the button is released, that is, when the desired rotation depth has been achieved, the upper housing portion is locked onto the lower housing portion to prevent accidental rotation or de-rotation.

In another exemplary implementation, base 12 comprises various shapes and flange 50 comprises various shapes complementary of base 12 for desired mating, such as substantially circular, substantially square, substantially rectangular, polygonal, or the like.

In another exemplary implementation, inflation medium 66 is a liquid.

In another exemplary implementation, inflation medium 66 is set at a predetermined temperature and then introduced into plenum 29.

In another exemplary implementation, inflatable bladder 20 is a disposable, primarily to combat transmission of infectious agents to subsequent patients.

In another exemplary implementation, inflatable bladder 20 is a non-disposable. As an example, a patient may use the same inflatable bladder 20 a number of times as recommended by a healthcare professional.

In yet another exemplary implementation, screw elements 38 may be rotatably attached to threaded posts 36 and positioned at different depths on threaded posts 36 in order to conform to the complex contours of anatomical structure 80. Accordingly, portions of flange 50 may be free of base 12. As inflatable bladder 20 expands it presses up against top wall 22 of housing 18 and presses down against wound 82 to apply the desired compressive force on wound 82, despite portions of flange 50 being free of base 12.

In yet another exemplary implementation, compression apparatus 10 comprises a housing integrally-formed with a base having a base opening with access to the wound. An inflatable member fillable with a fluid is included within the housing, and expandable within the housing and through the housing opening to apply a compressive force on the wound.

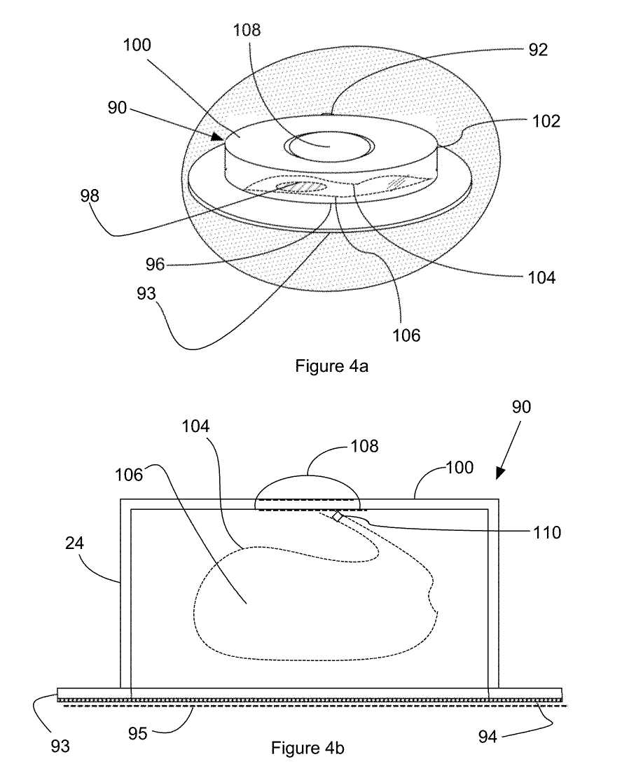

In yet another exemplary implementation, as shown in FIGS. 4a and 4b, compression apparatus 90 comprises housing 92 integrally-formed with base 93 with adhesive backing 94, and release liner 95. Base 93 also comprises base opening 96 which permits access to wound 98 on anatomical structure 99. Housing 92 further comprises top wall 100 and side wall 102 extending between top wall 102 and inflatable member 104 fillable with fluid 106 is included within housing 92, and expandable within housing 92 and through housing opening 96 to apply a compressive force on wound 98. Pump means 108 located on top wall 100 is coupled to inflatable member 104 and is configured to inflate inflatable member 104 with inflation medium 106. Generally, pump means 108 comprises a bulb pump having a fluid inlet and a fluid outlet in fluid communication with inflatable member 104. Pump means 108 also comprises at least one valve assembly 110 for permitting ingress and egress of inflation medium 106, such as a push pump, as is well known in the art.

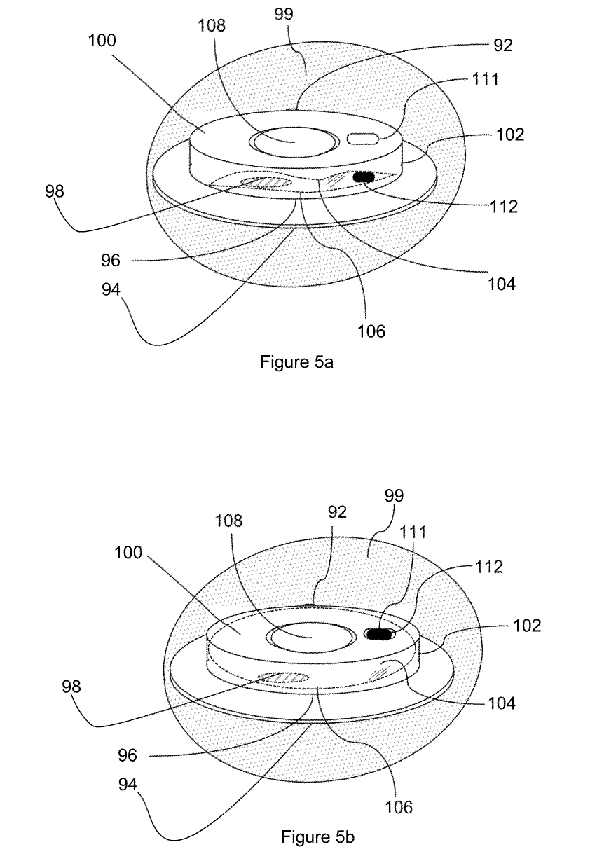

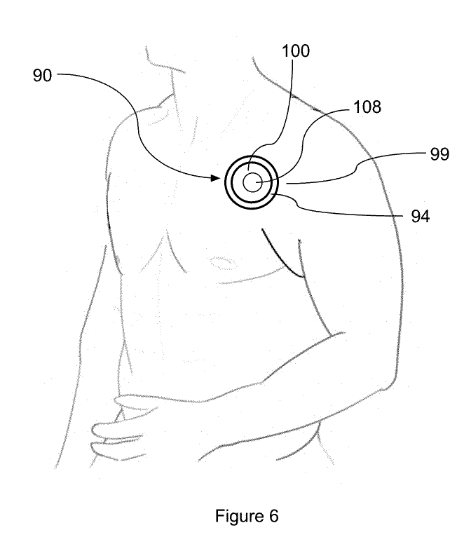

In yet another exemplary implementation, as shown in FIGS. 5a and 5b, top wall 102 comprises one or more ports 110 extending therethrough, and inflatable member 104 comprises indicia 112. Pump means 108 is caused to inflate inflatable member 104 with inflation medium 106 and transform the inflatable member 104 from a substantially deflated state into a substantially inflated state. As such, inflatable member 104 is caused to expand within housing 92 and through housing opening 96, and inflatable member 104 is expanded until indicia 112 becomes visible through port 111. At this instance, inflatable member 104 substantially abuts interior surface of top wall 102. Accordingly, the sight of indicia 112 though port 111 may be indicative that a desired or effective compressive force to be applied on wound 98 has been achieved. FIG. 6 shows compression apparatus 90 in use on a patient. As can be seen, compression apparatus 90 does not require straps or harnesses for securement on site 99 having wound 98.

While these exemplary embodiments are described in sufficient detail to enable those skilled in the art to practice the invention, it should be understood that other embodiments may be realized and that logical and mechanical changes may be made without departing from the spirit and scope of the invention. The preceding detailed description is presented for purposes of illustration only and not of limitation, and the scope of the invention is defined by the preceding description, and with respect to the attached claims.

1. A compression apparatus for a wound, the apparatus having:
a housing;
an inflatable member within the housing;
a base comprising a base opening with access to the wound; the base removably attached to the housing;
wherein the inflatable member is fillable with a fluid such that the inflatable member expands within the housing opening and expands through the base opening to apply a compressive force on the wound.

2. The compression apparatus of claim 1, further comprising a pump means for inflating the inflatable member, the pump means including a bulb pump having a fluid inlet and a fluid outlet in fluid communication with the inflatable member.

3. The compression apparatus of claim 2, wherein the pump means comprises at least one valve for permitting ingress and egress of the inflation medium.

4. The compression apparatus of claim 3, wherein the base comprises a peripheral portion defining the base opening.

5. The compression apparatus of claim 4, wherein the base comprises a top surface with a first fastening member and a bottom surface with an adhesive backing to removably attach the base around the wound such that the wound is located within the base opening.

6. The compression apparatus of claim 3, wherein the inflatable member is filled with the fluid at a predetermined pressure.

7. The compression apparatus of claim 3, further comprising a pressure indicator indicative of a predetermined operating pressure.

8. The compression apparatus of claim 3, wherein the inflation medium is at least one of a gas and a liquid.

9. The compression apparatus of claim 8, wherein the inflation medium is set to a predetermined temperature.

10. The compression apparatus of claim 4, wherein the base is integrally formed with the housing.

11. The compression apparatus of claim 3, wherein the housing comprises a top wall with a side wall depending therefrom to define a housing opening, and a flange extending from the side wall, and the flange having a second fastening member for cooperating with the first fastening member to removably attach the housing to the base.

12. The compression apparatus of claim 11, wherein the sidewall comprises a port extending therethrough, the port having an interior end coupled to an inflatable member and an exterior end coupled to a source of the inflation medium; wherein the port comprises a releasable valve.

13. The compression apparatus of claim 12, wherein the first fastening member and the second fastening member regulate the compressive force.

14. The compression apparatus of claim 4, wherein the housing comprises a lower housing portion comprising the flange and an upper housing portion rotatably attached to the lower housing portion.

15. The compression apparatus of claim 14, wherein the upper housing portion is rotated towards the lower housing to increase the compressive force or rotated away from the lower housing to decrease the compressive force, thereby regulating the compressive force on the wound.

16. A compression apparatus for a wound, the apparatus comprising:
a base comprising a base opening with access to the wound; wherein the base comprises a peripheral portion defining the base opening and a bottom surface with an adhesive backing to removably attach the base around the wound such that the wound is located within the base opening;
a housing comprising a top wall with a side wall depending therefrom to define a housing opening, and a flange extending from the side wall; and
an inflatable member within the housing; wherein the inflatable member is fillable with an inflation medium such that the inflatable member expands within the housing opening and expands through the base opening to apply a compressive force on the wound.

17. The compression apparatus of claim 16, wherein the base is removably attached to the base.

18. The compression apparatus of claim 16, further comprising pump means and at least one valve assembly for placing the inflatable member between an inflated state and a deflated state.

19. A method of applying a compression force on a wound, the method comprising:
providing a housing with an inflatable member within the housing, wherein the housing comprises a base with base opening for access to the wound;
removably attaching the base around the wound, wherein the base comprises an adhesive backing; and
filling the inflatable member with an inflation medium such that the inflatable member expands within housing and expands through the base opening to apply the compressive force on the wound.

20. The method of claim 19, wherein the inflatable member is associated with a pressure indicator.
